(19) United States
(12) Patent Application Publication
(10) Pub. No.: US 2010/0015209 A1
(43) Pub. Date: Jan. 21, 2010

(54) Patch and patch preparation

(75) Inventors: Miki Funahashi, Osaka (JP); Atsushi Hamada, Osaka (JP); Tsuyoshi Kasahara, Osaka (JP); Jun Ishikura, Osaka (JP)

Correspondence Address: WENDEROTH, LIND & PONACK, L.L.P., 1030 15th Street, N.W.,, Suite 400 East, Washington, DC, 20005-1503, US

(73) Assignee: Nitto Denko Corporation

(21) Appl. No.: 12/458,626

(22) Filed: Jul. 17, 2009

(30) Foreign Application Priority Data
Jul. 17, 2008 (JP) 186562/2008

(51) Int. Cl. A61K 9/70; B32B 7/12

(52) U.S. Cl. 424/447; 428/355 R

(57) ABSTRACT

Provided is a patch having good adhesion property, which stably maintains the quality for a long time.

A patch containing a support and an adhesive layer formed on at least one surface of the support, wherein the adhesive layer contains an elastomer having a structure of the formula I:

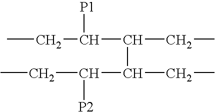

wherein P1 and P2 are each independently hydrogen, a hydroxy group or an organic group.

### TECHNICAL FIELD OF THE INVENTION

The present invention relates to a patch and a patch preparation. More particularly, the present invention relates to a patch and a patch preparation having a superior skin adhesion force, which leave less adhesive residue on the skin.

### BACKGROUND OF THE INVENTION

Patch is a convenient and effective dosage form for wound protection or administration of a drug to the body. The patch is requested to stably maintain the quality for a long time and have good adhesion property.

As a patch having such adhesion property, for example, patent document 1 and patent document 2 disclose patches having an adhesive layer comprised of a crosslinked rubber component having a functional group. In Example 4 of patent document 1, it is described with regard to such patch that oozing of an adhesive in 3 mm or less was observed in the peripheral area of a test piece. Thus, the patches are presumed to have insufficient cohesion force. Moreover, the patches of these documents may have unsatisfactory stability in patch quality due to the presence of the functional group in the rubber component. As shown above, none of the conventional patches sufficiently meet the above-mentioned requirements.
patent document 1: JP-A-03-127727
non-patent document 2: JP-A-10-151185

### DISCLOSURE OF THE INVENTION

### Problems to be Solved by the Invention

The present invention has been made in view of the above-mentioned situation and aims to provide a patch having good adhesion property and capable of stably maintaining the quality for a long time.

### Means of Solving the Problems

Accordingly, the present invention provides the following.
(1) A patch comprising a support and an adhesive layer formed on at least one surface of the support, wherein the adhesive layer comprises an elastomer having a structure of the formula I:

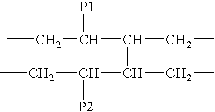

wherein P1 and P2 are each independently hydrogen, a hydroxy group or an organic group.
(2) The patch of (1), wherein the above-mentioned organic group is at least one selected from the group consisting of alkyl, alkoxy, aryl, aryloxy, acyl and acyloxy, each of which is optionally substituted.
(3) The patch of (1) or (2), wherein the above-mentioned organic group is at least one selected from the group consisting of phenyl, benzoyl and benzoyloxy.
(4) The patch of any of (1) to (3), wherein the above-mentioned adhesive layer further comprises an organic liquid component.
(5) The patch of any of (1) to (4), wherein the above-mentioned adhesive layer further comprises a tackifier.
(6) A patch preparation comprising the patch of any of (1) to (5) and a drug in the adhesive layer of the patch.

### Effect of the Invention

According to the present invention, a patch superior in the skin adhesion force, which leaves less adhesive residue on the skin and stably maintains the quality for a long time, can be obtained. Accordingly, the patch of the present invention is suitable for adhesion to the skin of mammals. In addition, when the patch of the present invention is used for, for example, a patch preparation, an undesirable reaction between a drug and an adhesive composition can be reduced while maintaining good adhesion property, and the quality of the drug can be stably maintained for a long time.

### BEST MODE OF CARRYING OUT THE INVENTION

A preferable embodiment of the present invention is shown below. The detailed explanation thereof and particular embodiment are solely intended for exemplification purposes and do not limit the scope of the present invention. The explanation of the preferable embodiment below is for exemplification purposes and does not intend to limit the present invention, application thereof and use thereof.

The patch of the present invention can employ a constitution comprising a support and an adhesive layer formed on at least one surface of the support. While the support is not particularly limited, a support substantially impermeable to the components in the adhesive layer, for example, adhesive, additive, drug and the like, namely, a support that does not permit them to pass through the support and be lost from the back face thereof, is preferable.

Examples of such support include single films of polyester, nylon, Saran (registered trade mark), polyethylene, polypropylene, polyvinyl chloride, ethylene-ethyl acrylate copolymer, polytetrafluoroethylene, Surlyn (registered trade mark), metal foil and the like, a laminate film thereof and the like. While the thickness of the support is not particularly limited, it is generally 10-500 μm, preferably 10-200 μm.

Among those, to improve the adhesion force (anchoring force) between the support and the adhesive layer, the support is preferably a laminate film of non-porous type plastic film recited above and a porous film. In this case, an adhesive layer is preferably formed on the porous film side.

As such porous film, one capable of improving the anchoring force with an adhesive layer is employed. Specifically, paper, woven fabric, non-woven fabric, knitted fabric, mechanically perforated sheet and the like can be mentioned. From the aspects of handling property and the like, paper, woven fabric and non-woven fabric are particularly preferable from among those.

In consideration of improvement of anchoring force, flexibility of the whole patch preparation and adhesion operability and the like, a porous film having a thickness of 10-200 μm is employed. In the case of a thin preparation, such as a plaster type and an adhesive tape type, one having a thickness of 10-100 μm is employed.

When woven fabric or non-woven fabric is used as a porous film, the fabric weight thereof is not particularly limited. It is generally 5-30 g/m², preferably 6-15 g/m².

In the present invention, the most preferable support is a laminate film of a 1.5-6 μm-thick polyester film (preferably, polyethylene terephthalate film) and a polyester (preferably, polyethylene terephthalate) non-woven fabric having a fabric weight of 6-12 g/m².

The adhesive layer contains an elastomer having a structure represented by the formula I:

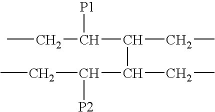

wherein P1 and P2 are each independently hydrogen, a hydroxy group or an organic group.

As shown in the formula I, the elastomer contained in the adhesive layer has a structure wherein two or more basic polymers having an unsaturated hydrocarbon chain are crosslinked by a carbon-carbon single bond. Since the structure contains crosslinking by a carbon-carbon single bond without using a functional group, the quality can be stably maintained for a long time during production and storage of the patch.

In the formula I, from the aspects of the stability of the quality of the adhesive layer, P1 and P2 are each independently selected from the group consisting of hydrogen, a hydroxy group and an organic group.

The above-mentioned “organic group” is, for example, at least one selected from the group consisting of alkyl, alkoxy, aryl, aryloxy, acyl and acyloxy, each of which is optionally partly substituted by other substituent. Among these, one free of substitution with a functional group such as hydroxy, carboxy, amino and the like is particularly preferable from the aspects of stabilization of the quality of the adhesive layer.

Here, examples of the “optionally substituted alkyl” include C₁₋₆ alkyl such as methyl, ethyl, n-propyl, sec-propyl, t-propyl, t-butyl, t-pentyl, t-hexyl and the like.

Examples of the “optionally substituted alkoxy” include C₁₋₆ alkoxy wherein the above-mentioned C₁₋₆ alkyl is bonded to an oxygen atom, and the like.

Examples of the “optionally substituted aryl” include C₆₋₁₀ aryl such as phenyl, naphthyl, cumyl and the like.

Examples of the “optionally substituted aryloxy” include C₆₋₁₀ aryloxy wherein the above-mentioned C₆₋₁₀ aryl is bonded to an oxygen atom such as phenyloxy, cumyloxy and the like.

Examples of the “optionally substituted acyl” include C₁₋₆ alkyl-carbonyl, C₆₋₁₀ aryl-carbonyl and the like such as benzoyl, isobutyryl, lauroyl, 2-ethylhexylmonocarbonate, 3-methylbenzoyl and the like.

In addition, examples of the “optionally substituted acyloxy” include C₁₋₆ alkyl-carbonyloxy, C₆₋₁₀ aryl-carbonyloxy and the like, wherein the above-mentioned “acyl” such as benzoyloxy and the like is bonded to an oxygen atom.

Of the above-mentioned organic groups, phenyl, benzoyl and benzoyloxy are particularly preferable from the aspects of easy availability and reactivity.

Here, the above-mentioned elastomer can be obtained by, for example, using a polymer having an unsaturated hydrocarbon chain as a basic polymer, eliminating a carbon-carbon double bond, which is an unsaturated bond, of the basic polymer in the presence of an organic peroxide, and crosslinking the eliminated part between basic polymers by a carbon-carbon single bond. Accordingly, introduction of a functional group into the basic polymer for crosslinking is not necessary, and various basic polymers can be selected without limitation by the introduced functional groups, thus increasing the degree of freedom in selection of the basic polymers. Moreover, the thus-obtained elastomer has a hydrocarbon chain having a comparatively high molecular weight, and can impart skin adhesion force and cohesion force necessary for a patch to the adhesive layer. In addition, the elastomer has a structure wherein hydrocarbon chains are intricately entangled three-dimensionally, and affords an elastomer having a high gel fraction. As a result, a cohesion force sufficient for a patch can be imparted to the adhesive layer.

While the basic polymer is not particularly limited, examples thereof include polymers having an unsaturated hydrocarbon chain represented by the formula II:

—CH₂—CH═CH—CH₂—

such as polybutadiene, styrene-butadiene block copolymer (SB), styrene-butadiene-styrene block copolymer (SBS) and the like. While the weight-average molecular weight of such polymer is not particularly limited, it is preferably 50,000-5,000,000. The weight-average molecular weight means a value measured by gel permeation chromatography under the following conditions. analysis conditions
GPC apparatus: HLCB120 (manufactured by Tosoh Corporation)
column: TSKgel GMH-H(S) (manufactured by Tosoh Corporation)
standard: polystyrene
eluent: tetrahydrofuran flow rate: 0.5 ml/min
measurement temperature: 40° C.
detector: differential refractometer

For example, when polybutadiene is used as a basic polymer, an elastomer having the structure of the formula I as a repeat unit, or an elastomer mainly containing the structure of the formula I, can be preferably obtained.

When styrene-butadiene block copolymer (SB) or styrene-butadiene-styrene block copolymer (SBS) is used as a basic polymer, an elastomer wherein styrene is connected to the structure of the formula I is obtained, thereby imparting a strong cohesion force to the adhesive layer.

In addition, the above-mentioned elastomer is preferably contained in the adhesive layer in a proportion of 10 wt %-80 wt %, particularly preferably 30 wt %-60 wt %, from the aspects of maintenance of cohesion force and the like.

Organic peroxide is not particularly limited, and those known per se which are generally used in the field of polymerchemical can be used. For example, diacyl peroxide (e.g., dibenzoyl peroxide, diisobutyryl peroxide, di(3,5,5-trimethylhexanoyl) peroxide, dilauroyl peroxide, disuccinic acid peroxide), peroxy esters (e.g., 1,1,3,3-tetramethylbutyl peroxy-2-ethylhexanoate, 2,5-dimethyl-2,5-di(2-ethylhexanoylperoxy)hexane), t-hexyl peroxy-2-ethylhexanoate, t-butyl peroxy-2-ethylhexanoate), ketone peroxide (e.g., methylethylketone peroxide), peroxyketal (e.g., 1,1-di(t-butylperoxy)cyclohexane), hydroperoxide (e.g., p-mentan hydroperoxide), dialkyl peroxide (for example, dicumyl peroxide), peroxydicarbonate (e.g., di-n-propylperoxydicarbonate) and the like can be mentioned.

Of these, from the aspects of reactivity, diacyl peroxide (particularly dibenzoyl peroxide) and peroxy ester (particularly 1,1,3,3-tetramethylbutyl peroxy-2-ethylhexanoate) are preferable. Particularly, diacyl peroxide is preferable, since it reacts with a basic polymer, and produces an elastomer wherein P1 and P2 are the same or different and each is alkyl, phenyl, acyl, benzoyl, acyloxy or benzoyloxy, each of which is optionally substituted. Alternatively, peroxy ester produces an elastomer wherein P1 and P2 are the same or different and each is alkyl, alkoxy, phenyl, acyl, benzoyl, acyloxy or benzoyloxy, each of which is optionally substituted. The content of organic peroxide is preferably 0.1-5 parts by weight per 100 parts by weight of the basic polymer. When the content of organic peroxide is less than 0.1 part by weight per 100 parts by weight of the basic polymer, the cohesion force may become insufficient, and when it exceeds 5 parts by weight, the adhesive layer becomes hard and the adhesive force may become insufficient.

As mentioned above, an elastomer having a structure of the formula I of the present invention has a structure wherein a hydrocarbon chain is intrinsically entangled. An adhesive layer containing such an elastomer has a sufficient cohesion force. Therefore, the patch of the present invention can contain a large amount of other components, such as a tackifier and the like in the adhesive layer. As a result, the patch of the present invention can more improve adhesive force and tackiness during adhesion to the skin. On the other hand, when the patch of the present invention is adhered to the skin and later detached, the possibility of leaving the adhesive layer on the skin surface is small.

As the tackifier, those known in the field of patch is appropriately selected and used. Examples of the tackifier include petroleum resin (e.g., aromatic petroleum resin, aliphatic petroleum resin), terpene resin, rosin resin, coumaroneinden resin, styrene resin (e.g., styrene resin, α-methylstyrene), hydrogenated petroleum resin (e.g., alicyclic saturated hydrocarbon resin) and the like. Among these, alicyclic saturated hydrocarbon resin is preferable since the preservation stability of the drug becomes fine.

Tackifiers can be used in a combination of one or more kinds thereof. When two or more kinds are used in combination, for example, resins having different kinds and softening points may be combined.

The content of tackifier is preferably 10-180 parts by weight, more preferably 30-90 parts by weight, per 60 parts by weight of the basic polymer. When the content of tackifier is less than 10 parts by weight, the tackiness and cohesion force may become poor, and when it exceeds 180 parts by weight, the adhesive layer tends to be too soft and sticky.

In addition, in the patch of the present invention, since the adhesive layer has a sufficient cohesion force as mentioned above, the adhesive layer can contain a large amount of organic liquid components. As a result, the patch of the present invention can provide a soft feeling upon adhesion to the skin, and low irritation upon detachment from the skin. In this case, even when the patch of the present invention is released after adhesion to the skin, the possibility of the adhesive layer remaining on the skin surface is small. In addition, when the adhesive layer contains the below-mentioned drug, the transdermal absorption thereof can be promoted.

As the organic liquid component, a hydrophobic liquid component is preferable, such as fatty acid alkylester, from the aspect of compatibility with the adhesive layer.

Examples of the fatty acid alkylester include fatty acid alkylester comprised of higher fatty acid having 12-16 carbon atoms, preferably 12-14 carbon atoms, and lower monovalent alcohol having 1-4 carbon atoms. The above-mentioned higher fatty acid includes lauric acid (C12), myristic acid (C14) or palmitic acid (C16), preferably myristic acid. Examples of the above-mentioned monovalent alcohol include methyl alcohol, ethyl alcohol, propyl alcohol, butyl alcohol and the like, preferably isopropyl alcohol. Accordingly, preferable fatty acid alkyl ester is isopropyl myristate. The organic liquid components may be used in combination of one or more kinds thereof.

The content of organic liquid component is preferably 3-240 parts by weight, more preferably 4-150 parts by weight, per 60 parts by weight of the basic polymer. When the content of organic liquid component is less than 3 parts by weight, a superior soft feeling is imparted to the adhesive layer and, when the adhesive layer contains a drug, a high transdermal proabsorptive effect can be obtained. When it is not more than 240 parts by weight, a superior soft feeling is imparted to the adhesive layer and, when the adhesive layer contains a drug, a high transdermal proabsorptive effect can be advantageously obtained while suppressing a decrease in the adhesive force and cohesion force of the whole adhesive layer.

The adhesive layer may further contain other additives known per se, for example, anti-aging agent, antioxidant, UV absorber, filler and the like, as necessary.

The thickness of the adhesive layer is preferably 10 μm-1000 μm, particularly preferably 20 μm-500 μm, from the aspects of skin adhesiveness.

The present invention also relates to a patch preparation containing a drug in the adhesive layer. As mentioned above, the patch of the present invention contains an elastomer having a structure of the formula I in the adhesive layer, and has a structure-wherein two or more basic polymers are crosslinked by a carbon-carbon single bond without using a functional group. As a result, the need to consider an undesired reaction of the functional group and the drug can be reduced, thus increasing the degree of freedom in the design of the patch preparation. In addition, since the number of carbon-carbon double bonds in the unsaturated hydrocarbon chain of the basic polymer decreases due to the carbon-carbon single bond between basic polymers, the reaction points decreases, thus possibly contributing to the stability of the drug.

The drug here is not particularly limited, and one permitting administration to mammals such as human and the like through the skin thereof, i.e., a transdermally absorbable drug is preferable. Specific examples of such drug include general anesthetics, hypnotic sedatives, antiepileptic drugs, antipyretic analgesic antiphlogistic drugs, anti-vertiginous drugs, psychoneurotic drugs, topical anesthetics, skeleton muscle relaxants, autonomic drugs, antiepileptic drugs, anti-parkinsonian drugs, anti-histamine drugs, cardiac stimulants, drugs for arrhythmia, diuretics, hypotensive drugs, vasoconstrictors, coronary vasodilators, peripheral vasodilators, arteriosclerosis drugs, drugs for circulatory organ, anapnoics, antitussive expectorants, hormone drugs, external drugs for mattery diseases, analgesic•antipruritic•styptic•antiphlogistic drugs, drugs for parasitic dermatic diseases, drugs for arrest of bleeding, gout treatment drugs, drugs for diabetes, drugs for anti-malignant tumor, antibiotics, chemical therapy drugs, narcotics, quit smoking aids and the like.

While the content of the drug is not particularly limited as long as it satisfies an effect of transdermally absorbable drugs and does not impair the adhesion property of the adhesive, it is, for example, 0.5-50 parts by weight per 60 parts by weight of the basic polymer.

In addition, the patch and patch preparation of the present invention can be produced by any method, for example, the following methods.
[1] A method comprising (i) a step of dissolving or dispersing a basic polymer containing an unsaturated hydrocarbon chain and, where necessary, a tackifier, an organic liquid component, a drug and the like in a solvent, adding organic peroxide and mixing and stirring the mixture; (ii) a step of applying the obtained adhesive solution or dispersion to at least one surface of a support, and drying to form the adhesive layer on the surface of the support; and (iii) a step of forming a release liner on the adhesive layer (i.e., direct coating).
[2] A method comprising (i) a step of applying the above-mentioned adhesive solution or dispersion to at least one surface of a release liner for protection; (ii) a step of drying to form the adhesive layer on the surface of a release liner; (iii) a step of adhering a support to the adhesive layer (i.e., indirect coating).

To promote crosslinking of the basic polymer in the adhesive layer, the above-mentioned methods [1] and [2] preferably further include, for example, a maturing step. A maturing step can be performed by, for example, heating and preserving at 50-300° C. for 10-100 hr.

### EXAMPLES

The present invention is explained in more detail in the following by referring to Examples, which are not to be construed as limitative.

### 1. Starting Materials of Adhesive Layer

basic polymer containing unsaturated hydrocarbon chain: BR (polybutadiene) weight-average molecular weight 458,000 basic polymer free of unsaturated hydrocarbon chain: PIB (polyisobutylene) weight-average molecular weight 425,000 organic peroxide (PO): dibenzoyl peroxide tackifier: alicyclic saturated hydrocarbon resin (softening point 100.5° C.) organic liquid component: isopropyl myristate

### 2. Production of Elastomer in Flask

Polybutadiene or polyisobutylene and organic peroxide (dibenzoyl peroxide) were blended at a ratio shown in Table 1 in toluene as a solvent, mixed and heated at 120° C. for 4 hr to give an elastomer. The tensile strength of the obtained elastomer was measured as follows.

### Measurement Method

An elastomer solution was applied onto a release liner such that the thickness after drying was 100 μm, and dried to give an adhesive sheet. The adhesive sheet was cut into a rectangle having width 20 mm, length 40 mm to give a sample. The sample was densely wound from one side in the length direction of the rectangle with the side as the central axis to give a cylindrical or round-stick test piece. The stress of the test piece was measured at chuck distance 20 mm, tensile strength 300 mm/min using a tensile tester (manufactured by SHIMADZU Corporation, AUTOGRAPH AG-IS), and the maximum stress up to rupture was compared. The results are shown in Table 1.

As is clear from Table 1, the maximum stress markedly increased in BR as compared to PIB in the presence of PO. The maximum stress of PIB decreased in the presence of PO. The remarkable difference in the maximum stress shows that BR was crosslinked by a carbon-carbon single bond in the presence of PO.

### 3. Patch

### Examples 1-4, Comparative Examples 1, 2

An adhesive solution having a solid content of 20-40 wt % was prepared using toluene as a solvent and at the mixing ratio shown in Table 2. This was uniformly applied to a polyethylene terephthalate film as a release liner such that the thickness after drying the adhesive layer became 100 μm, and dried at 100° C. for 5 min. Using a laminate of a polyethylene terephthalate non-woven fabric and a polyethylene terephthalate film as a support, the surface of the polyethylene terephthalate non-woven fabric and the adhesive layer were faced to each other and adhered to give a laminate. The prepared laminate was aged by heating and storing in the presence of nitrogen to give a patch.

### 4. Test Method

The patches of Examples 1-4 and Comparative Examples 1, 2 were measured as follows regarding the following evaluation items.

### (1) Adhesive Force

In a room at 23° C., 60% RH, the patch was cut into a test piece having a width 12 mm, length 5 cm, a release liner of the test piece was removed, and a test piece was press-adhered to a phenol resin plate (test plate) by one reciprocation of a 2 kg roller. The plate was stood for 30 min under this environment, and the adhesive force of the test piece was measured by a tensile tester by stretching the test piece at a releasing angle of 180° and a releasing rate of 300 mm/minute. In the destruction mode, cohesive failure was G and interface destruction was K. Here, a patch having an adhesive force of not less than 0.2 N/12 mm and free of cohesive failure is preferable.

### (2) Retaining Force

In a room at 23° C., 60% RH, the patch was cut into a test piece having a width 10 mm, length 5 cm, a release liner of the test piece was removed, and a test piece was press-adhered to a phenol resin plate (test plate) by one reciprocation of a 2 kg roller. The adhered area then was set to 200 mm². The plate was stood for 20 min under 40° C. environment and, under 40° C. environment, the surface of the test plate, to which the test piece was not adhered, was fixed so that the test plate would be perpendicular to the floor, a 300 g load was hung from the test piece and the time up to a fall of the test piece from the test plate was evaluated as retaining force. In the destruction mode, cohesive failure was G, interface destruction was K, and partial cohesive failure was K/G. Here, a patch having a retaining force that does not permit a fall in a short time is preferable.

From the results of Table 2, the patches of Examples 1-4 have good adhesive force and good cohesion force as the adhesive layer of patch. Particularly, the patch of Example 2 was superior in the adhesive force, and had an appropriate cohesion force due to its retaining force and preferable property as a patch. In addition, the patches of Examples 1-4 showed interface destruction between the test plate and the adhesive layer of the patch during the adhesive force measurement, and an adhesive residue leaving an adhesive on the test plate was not observed.

In contrast, Comparative Example 1 and Comparative Example 2 showed cohesive failure during the adhesive force measurement, and an adhesive residue leaving an adhesive on the test plate was observed. Also, the cohesion force was also insufficient, and the test piece fell in a short time during the measurement of retaining force, and cohesive failure occurred during the fall of the test piece. They were considered to be attributable to the absence of addition of organic peroxide in Comparative Example 1, which prevented crosslinking of basic polymers having an unsaturated double bond by a carbon-carbon single bond, resulting in insufficient cohesion force. It is considered that since PIB free of a carbon-carbon double bond was used as the basic polymer in Comparative Example 2, the basic polymers were not crosslinked by a carbon-carbon single bond, or crosslinking was insufficient to result in insufficient cohesion force.

### 5. Patch Preparation

### Examples 5-8

In the same manner as in the above-mentioned Examples 1-4, except that 1 part by weight is subtracted from 35-60 parts by weight of each basic polymer in Examples 1-4 and 1 part by weight of indomethacin is added as a drug, the patch preparations of Examples 5-8 are prepared.

The patch preparations of Examples 5-8 have the properties similar to those of the patches of the above-mentioned Examples 1-4.

The explanation of the present invention is simply of an exemplification nature, where a modified embodiment within the gist of the present invention is intended to be within the scope of the present invention. Such a modified embodiment should not be considered to deviate from the spirit and scope of the present invention.

TABLE 1

| | composition | | | | | |
|---|---|---|---|---|---|---|
| | | | | | | amount (parts |
| | | | | | organic | by weight) of |
| | | | basic | | liquid | organic |
| | | basic | polymer | | component | peroxide per |
| | | polymer:tackifier | (parts | tackifier | (parts by | 100 parts by |
| | basic polymer | (weight | by | (parts by | weight) | weight of |
| | kind | ratio) | weight) | weight) | IPM | basic polymer |
| Ex. 1 | polybutadiene | 1:1 | 35 | 35 | 30 | 0.2 |
| Ex. 2 | polybutadiene | 1:1 | 35 | 35 | 30 | 0.3 |
| Ex. 3 | polybutadiene | 1:1 | 35 | 35 | 30 | 0.35 |
| Ex. 4 | polybutadiene | 1:1 | 35 | 35 | 30 | 0.4 |
| Ex. 5 | polybutadiene | 3:1 | 52.5 | 17.5 | 30 | 0.3 |
| Ex. 6 | polybutadiene | 3:1 | 52.5 | 17.5 | 30 | 0.4 |
| Ex. 7 | polybutadiene | 2:1 | 46.7 | 23.3 | 30 | 0.3 |
| Ex. 8 | polybutadiene | 1:1.25 | 31.1 | 38.9 | 30 | 0.3 |
| Ex. 9 | polybutadiene | 1:1.5 | 28.0 | 42.0 | 30 | 0.3 |
| Ex. 10 | polybutadiene | 3:1 | 60 | 20 | 20 | 0.2 |
| Comp. | polybutadiene | 3:1 | 60 | 20 | 20 | 0 |
| Ex. 1 | | | | | | |
| Comp. | polyisobutylene | 3:1 | 60 | 20 | 20 | 1 |
| Ex. 2 | | | | | | |

| | measurement | | | | | |
|---|---|---|---|---|---|---|
| | adhesive force [N/12 mm] | | | adhesion [min] | | |
| | average | destruction | evaluation | average | destruction | evaluation |
| Ex. 1 | 1.30 | K | ⊙ | 5.17 | K/G | ⊙ |
| Ex. 2 | 0.72 | K | ⊙ | 7.40 | K/G | ⊙ |
| Ex. 3 | 0.64 | K | ⊙ | 10.10 | K/G | ⊙ |
| Ex. 4 | 0.55 | K | ◯ | 24 h< | — | ◯ |
| Ex. 5 | 0.14 | K | Δ | 24 h< | — | ◯ |
| Ex. 6 | 0.16 | K | Δ | 24 h< | — | ◯ |
| Ex. 7 | 1.42 | K | ⊙ | 103.10 | K/G | ⊙ |
| Ex. 8 | 0.80 | K | ⊙ | 2.70 | K/G | ⊙ |
| Ex. 9 | 0.83 | K | ⊙ | 2.90 | K/G | ⊙ |
| Ex. 10 | 1.59 | K | ⊙ | 24 h< | — | ◯ |
| Comp. | 1.00 | G | X | 9.90 | G | X |
| Ex. 1 | | | | | | |
| Comp. | 1.74 | G | X | 1.27 | G | X |
| Ex. 2 | | | | | | |

TABLE 2

| | | organic peroxide (parts by weight) | | | organic | | |
|---|---|---|---|---|---|---|---|
| | basic | | amount (parts by | | liquid | adhesive | |
| | polymer/amount | amount | weight) per 100 parts | tackifier | component | force (N/12 mm)/ | |
| | (parts by | (parts by | by weight of basic | (parts by | (parts by | destruction | adhesion (min)/ |
| | weight) | weight) | polymer | weight) | weight) | mode | destruction mode |
| Ex. 1 | BR/60 | 0.6 | 1 | 20 | 20 | 0.79/K | did not fall for 24 hr |
| | | | | | | | or longer |
| Ex. 2 | BR/35 | 0.0875 | 0.25 | 35 | 30 | 0.92/K | 5.2/K/G |
| Ex. 3 | BR/35 | 0.175 | 0.5 | 35 | 30 | 0.59/K | did not fall for 24 hr |
| | | | | | | | or longer |
| Ex. 4 | BR/35 | 0.35 | 1 | 35 | 30 | 0.48/K | did not fall for 24 hr |
| | | | | | | | or longer |
| Comp. | BR/60 | 0 | 0 | 20 | 20 | 1/G | 9.9/G |
| Ex. 1 | | | | | | | |
| Comp. | PIB/60 | 0.6 | 1 | 20 | 20 | 1.74/G | 1.27/G |
| Ex. 2 | | | | | | | |

### INDUSTRIAL APPLICABILITY

The patch of the present invention stably maintains the quality for a long time, is superior in the skin adhesion force, is associated with less adhesive residue on the skin surface, and can be utilized for a patch preparation.

This application is based on a patent application No. 2008-186562 filed in Japan (filing date: Jul. 17, 2008), the contents of which are incorporated in full herein by this reference.

1. A patch comprising a support and an adhesive layer formed on at least one surface of the support, wherein the adhesive layer comprises an elastomer having a structure of the formula I: 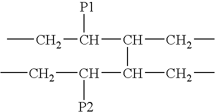
wherein P1 and P2 are each independently hydrogen, a hydroxy group or an organic group.

2. The patch of claim 1, wherein the organic group is at least one selected from the group consisting of alkyl, alkoxy, aryl, aryloxy, acyl and acyloxy, each of which is optionally substituted.

3. The patch of claim 1, wherein the organic group is at least one selected from the group consisting of phenyl, benzoyl and benzoyloxy.

4. The patch of claims 1, wherein the adhesive layer further comprises an organic liquid component.

5. The patch of claim 1, wherein the adhesive layer further comprises a tackifier.

6. A patch preparation comprising the patch of claim 1 and a drug in the adhesive layer of the patch.

7. The patch of claim 2, wherein the organic group is at least one selected from the group consisting of phenyl, benzoyl and benzoyloxy.

8. The patch of claim 2, wherein the adhesive layer further comprises an organic liquid component.

9. The patch of claim 3, wherein the adhesive layer further comprises an organic liquid component.

10. The patch of claim 2, wherein the adhesive layer further comprises a tackifier.

11. The patch of claim 3, wherein the adhesive layer further comprises a tackifier.

12. The patch of claim 4, wherein the adhesive layer further comprises a tackifier.

13. A patch preparation comprising the patch of claim 2 and a drug in the adhesive layer of the patch.

14. A patch preparation comprising the patch of claim 3 and a drug in the adhesive layer of the patch.

15. A patch preparation comprising the patch of claim 4 and a drug in the adhesive layer of the patch.

16. A patch preparation comprising the patch of claim 5 and a drug in the adhesive layer of the patch.
